(19) United States
(12) Patent Application Publication
(10) Pub. No.: US 2018/0256160 A1
(43) Pub. Date: Sep. 13, 2018

(54) LOADING FIXTURE FOR A SUTURING DEVICE AND METHOD FOR LOADING SAME

(71) Applicant: Dura Tap LLC, Wayne, PA (US)

(72) Inventors: Mark F. Kurd, Bryn Mawr, PA (US); David Greg Anderson, Moorestown, NJ (US); Jens Johnson, Austin, TX (US); Jay Tapper, Wayne, PA (US)

(21) Appl. No.: 15/454,093

(22) Filed: Mar. 9, 2017

(51) Int. Cl. A61B 17/04; A61B 17/06

(52) U.S. Cl. CPC A61B 17/0491; A61B 17/0469; A61B 17/06133; A61B 17/06066; A61B 2017/0608; A61B 2017/0472; A61B 2017/06052; A61B 2017/061; A61B 2017/0479

(57) ABSTRACT

A loading fixture for loading a needle into a suturing device includes a chassis, a clamp assembly and a needle holder. The clamp assembly is connected with the chassis. The needle holder is movably mounted to the chassis. A method of assembling a suturing device includes holding a suturing device. The method further includes inserting a needle having a suture attached thereto through a distal opening into a needle passage of the suturing device. The method also includes maintaining a portion of the suture extending out of the distal opening and outside of the suturing device.

### BACKGROUND

The present disclosure relates generally to surgery and the placement of sutures, and more particularly, to devices and methods for the suture repair of tissue, for example, tears of the dura mater that occur during spinal surgery.

Tears of the dura mater (durotomy) are a relatively common occurrence during spinal surgery. Incidences of durotomy can vary by procedure and can be an additional challenge during surgical repairs such as, for example, lumbar surgeries or the like. Moreover, it is desirable to form a substantially watertight closure of the dura mater to inhibit or preclude, for example, cerebrospinal fluid (CSF) leaks that can otherwise lead to patient complications.

Surgical closure techniques using sutures is one approach to dural repair. In some instances, however, these techniques can be difficult to execute due to anatomic constraints, obstruction of visualization by CSF or blood, and the proximity to nerve rootlets. In some instances, these challenges can be further complicated when using minimally invasive techniques such as, for example, a tubular retractor. In some such instances, surgeons may choose not repair the durotomy or they may attempt to repair the durotomy using traditional suturing tools. Such tools and devices can be limited and, in some instances, lack maneuverability to avoid obstructions and/or to enable adequate passage of the needle and suture through the tissue. As a result, surgical repairs of the dura mater are often time consuming and expensive.

Assembly of a suturing device for use in the aforementioned surgical closure techniques can present challenges.

### SUMMARY

In view of the foregoing, a loading fixture for loading a needle into a suturing device includes a chassis, a clamp assembly and a needle holder. The clamp assembly is connected with the chassis. The needle holder is movably mounted to the chassis.

Also, a method of assembling a suturing device includes holding a suturing device. The method further includes inserting a needle having a suture attached thereto through a distal opening into a needle passage of the suturing device. The method also includes maintaining a portion of the suture extending out of the distal opening and outside of the suturing device.

### BRIEF DESCRIPTION OF THE DRAWINGS

FIG. 1 is a perspective view of a suturing device.

FIG. 2 is a side view of the suturing device.

FIG. 3 is a cross-sectional view of a lower portion of the suturing device of FIG. 1 and a schematic depiction of a tissue tear.

FIG. 4 is another cross-sectional view of the lower portion of the suturing device of FIG. 1 and FIG. 4A is an enlarged view of the circled portion of FIG. 4.

FIG. 5 is another cross-sectional view of the lower portion of the suturing device of FIG. 1.

FIG. 6 is a perspective view of a loading fixture and the suturing device of FIG. 1 with a slide of the loading fixture in a first position.

FIG. 7 is an enlarged perspective view the loading fixture and the suturing device shown in FIG. 6.

FIG. 8 is a perspective view of the loading fixture and the suturing device of FIG. 1 with the slide of the loading fixture in a second position.

### DETAILED DESCRIPTION

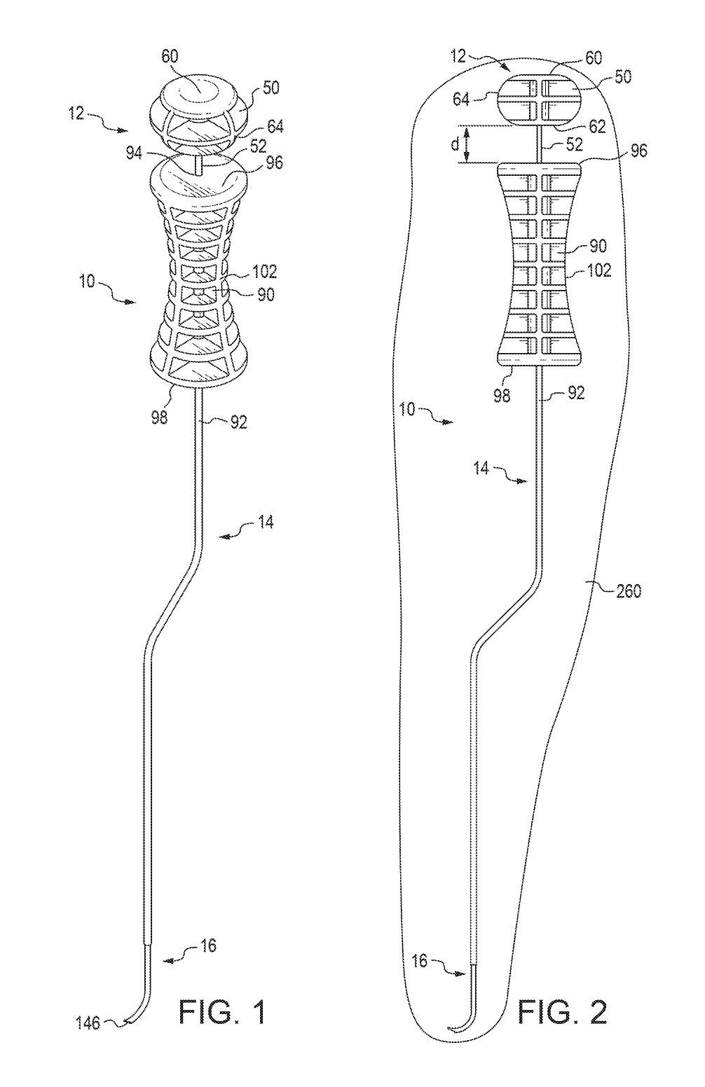

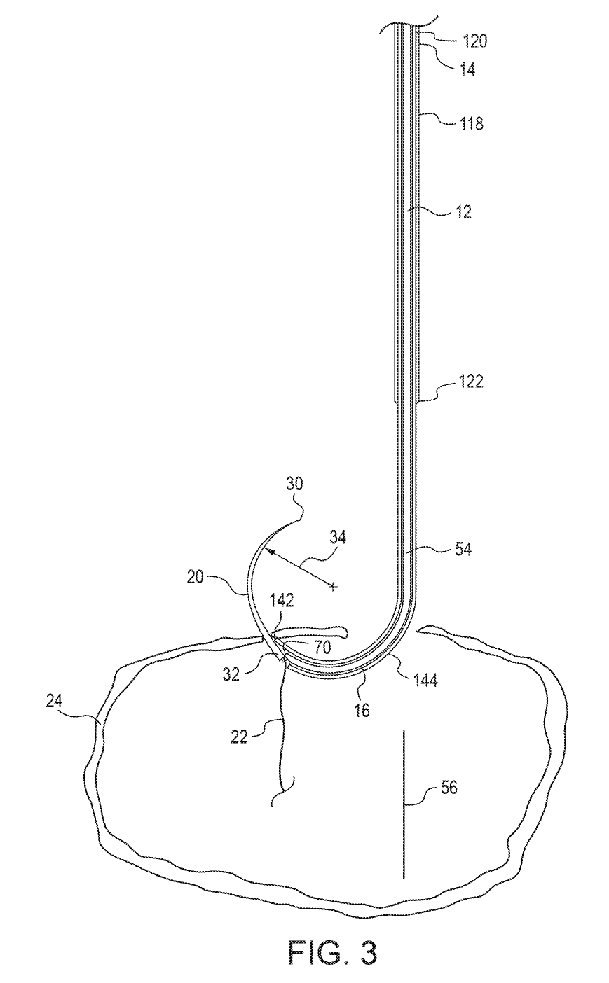

FIGS. 1 and 2 depict an example of a suturing device 10 that is useful to suture tears in dura mater, which may occur during spinal surgery procedures; however, the suturing device 10 can be used in other types of surgical procedures. The suturing device 10 generally includes an actuator 12, an elongate body 14, and a needle holder 16. The suturing device 10 is particularly useful during a minimally invasive surgical procedure that is performed through a tubular retractor or other small surgical portal to accurately locate a needle 20 and a suture 22, which are shown in FIG. 3, with respect to target tissue 24 to be sutured. The target tissue 24 shown in FIG. 3 is part of a dural sac having a tear. Again, the suturing device 10 may be useful in other surgical procedures.

With reference to FIG. 3, the needle 20 in the illustrated embodiment is a curved needle having a first end 30, which is pointed, and a second end 32, which is opposite to the first end 30. The needle 20 can be similar to commercially available curved needles made from known materials. The needle 20 can be formed having a curved needle radius 34. The needle 20 could also be formed from a malleable, or flexible, material such that the needle 20 could follow a curve when positioned within the needle holder 16, which is curved in FIG. 3, and then later straighten after exiting the needle holder 16. The needle 20 can take other configurations, such as straight.

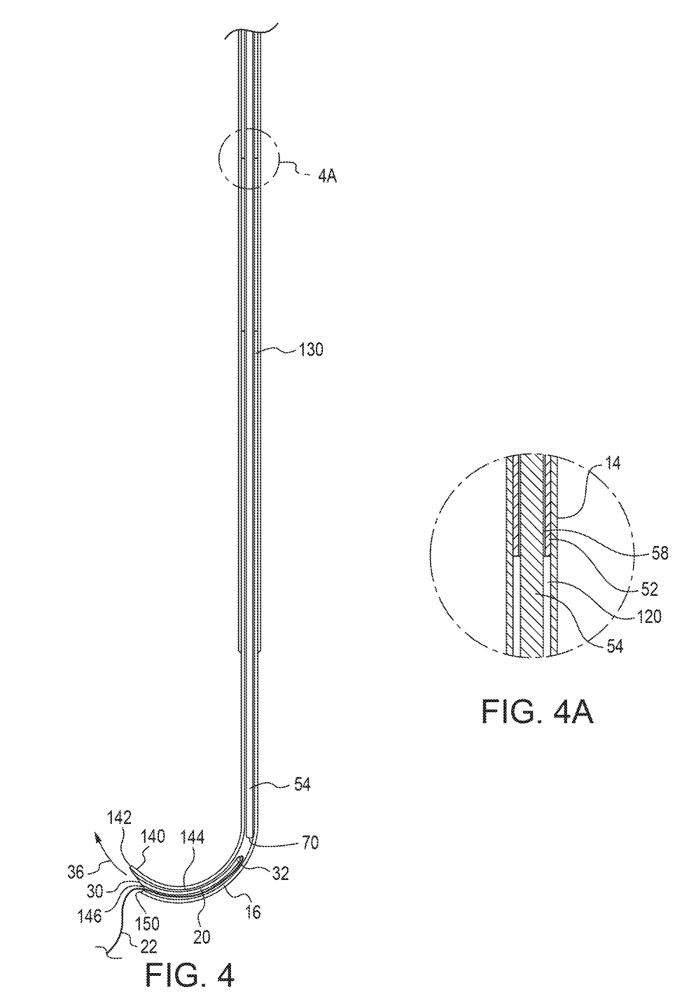

Actuation of the actuator 12 moves the needle 20 in an advance direction 36 (FIG. 4) with respect to the needle holder 16. The needle 20 moves from a retracted position, which is shown in FIG. 4, to a released condition, which is shown in FIG. 3, in which the needle 20 is released from the needle holder 16. When in the released condition, the surgeon can grasp the needle 20, for example with forceps, and pull the needle 20 and the suture 22.

With reference back to FIG. 3, the suture 22 connects with the needle 20 and extends from the second end 32 of the needle 20. The suture 22 can be swaged to the second end 32 of the needle 20. The suture 22 can also connect with the needle 20 in other conventional manners. The suture 22 can be acquired from known suture manufacturers.

The actuator 12 is operable between a first operating position and a second operating position. The actuator 12 in the illustrated embodiment is moveable between a first operating position, which is shown in FIG. 1, and a second operating position, which is shown in FIG. 4. Movement of the actuator 12 from the first operating position toward the second operating position moves the needle 20 in the advance direction 36 (FIG. 4) with respect to the needle holder 16 thus moving the needle 20 toward the released condition, which is shown in FIG. 3, in which the needle 20 is released from the needle holder 16.

With reference back to FIG. 1, in the illustrated embodiment the actuator 12 includes a button 50, a tube 52, which could also be a rod, and a wire 54 (FIG. 3). In the illustrated embodiment, the button 50 connects with the tube 52, which is connected with the wire 54. Alternatively, the button 50 could connect with the wire 54 without the tube 52. Also, the button 50 could connect with a rod having no elongate passage, and the rod can connect with the wire 54. In the illustrated embodiment, the actuator 12 includes a flexible section, which in the illustrated embodiment is made up of the wire 54, which can be made from nitinol. The flexible section is configured to bend within the needle holder 16 when the actuator 12 is moved from the first operating position toward the second operating position.

The tube 52 (or rod) is received within the elongate body 14 and moves with respect to the elongate body 14 when the actuator 12 moves between the first operating position and the second operating position. In the illustrated embodiment, the tube 52 moves along a longitudinal axis 56 (FIG. 3). The longitudinal axis 56 in the illustrated embodiment is a straight line; however, the longitudinal axis could be a curved line, for example if the elongate body 14 is curved. The tube 52 includes an elongate passage 58 (FIG. 4A), which receives the wire 54 in the illustrated embodiment. Alternatively, the wire 54 could extend from a distal end of a rod, which would connect with the button 50, in lieu of providing the tube 52. The tube 52 is made from a rigid material, such as a rigid plastic or metal, and is more rigid than the wire 54.

With reference back to FIG. 1, the button 50 includes an operator contact surface 60 that is configured to be depressed by a surgeon's finger or thumb to move the actuator 12 from the first operating position toward the second operating position. The button 50 also includes a handle contact surface 62 spaced from the operator contact surface 60 along the longitudinal axis 56. The button 50 also includes an outer surface 64, which follows a surface of revolution about the longitudinal axis 56 and spans between the operator contact surface 60 and the handle contact surface 62, which allows for the surgeon to easily manipulate the suturing device 10 and rotate the suturing device 10 about the longitudinal axis 56. The button 50 connects with the tube 52 (or the rod) and the wire 54 such that movement of the button 50 along the longitudinal axis 56 results in movement of the tube 52 (or rod) and the wire 54 along the longitudinal axis 56.

The suturing device 10 also includes a handle 90 connected with the elongate body 14. The handle 90 connects with a proximal end portion 92 of the elongate body 14 and is fixed to the elongate body 14 such that movement of the handle 90, e.g., rotational or translational movement, results in the same movement of the elongate body 14. The handle 90 includes an elongate bore 94 in which the proximal end portion 92 of the elongate body 14 is received. The elongate bore 94 extends from a proximal end surface 96 to a distal end surface 98 and is aligned with the longitudinal axis 56. The handle 90 also defines an outer side surface 102 extending between the proximal end surface 96 and the distal end surface 98. The outer side surface 102 follows a surface of revolution about the longitudinal axis 56. In the illustrated embodiment, the outer side surface 102 is a hyperboloid.

The button 50 is offset from the handle 90 when the actuator 12 is in the first operating position. More particularly, the handle contact surface 62 of the button 50 is offset from the proximal end surface 96 of the handle 90 a distance d as measured parallel to the longitudinal axis 56. The distance d can be configured such that the distal end 70 of the actuator 12 remains inside the needle holder 16 when the actuator 12 is in the second operating position, which can be when the handle contact surface 62 of the button 50 contacts the proximal end surface 96 of the handle 90.

The elongate body 14 in the illustrated embodiment is in the form of a cannula. With reference to FIG. 3, the elongate body 14 has an outer surface 118 (FIG. 3), which is smooth, and defines a track 120 that receives a portion of the actuator 12. In the illustrated embodiment, the elongate body 14 is a cannula and the track 120 is a lumen that receives the tube 52 and the wire 54 of the actuator 12. In the illustrated embodiment, the elongate body 14 is circular in a cross section taken normal to the longitudinal axis 56, however, the elongate body 14 could take alternative configurations, such as polygonal or U-shaped in a cross section taken normal to the longitudinal axis 56. The elongate body 14 includes the proximal end portion 92 and a distal end portion 122. In the illustrated embodiment, the needle holder 16 is received in and connected with the elongate body 14 and extends away from the distal end portion 122. Alternatively, the needle holder 16 can be provided as part of the distal end portion 122 of the elongate body 14. In the illustrated embodiment, the elongate body 14 is made from metal and extends along the longitudinal axis 56. The elongate body 14 in the illustrated embodiment is made from a rigid material; however, if desired at least a portion of the elongate body 14 may be made from a malleable or flexible material to allow the surgeon to bend at least a portion of the elongate body 14 into a desirable configuration for insertion into an animal body during a surgical procedure. The outer diameter of the elongate body 14 can be less than 3.5 mm, which provides a very slim device to enhance the line of sight for a surgeon during the surgical procedure.

The needle holder 16 extends away from the distal end portion 122 or is provided as part of the distal end portion 122 of the elongate body 14. With reference to FIGS. 3 and 4, the needle holder 16 is a hollow tubular member. In the illustrated embodiment, a proximal end section 130 of the needle holder 16 that is aligned with the longitudinal axis 56 is received inside the elongate body 14; however, the needle holder 16 could be formed as part of the elongate body, e.g., both the elongate body 14 and the needle holder 16 could be made from one tubular stock material. The needle holder 16 depicted in the illustrated embodiment is a curved needle holder that generally follows a constant radius such that the suturing device 10 can have J-hook configuration at a distal end thereof. In the illustrated embodiment, the needle holder 16 is not intended to be removable from the elongate body 14; however, in an alternative arrangement the needle holder 16 can selectively connect with the elongate body 14 via a mechanical connection such as a friction fit or a bayonet connection.

With reference to FIG. 4, the needle holder 16 includes a distal end section 140 having a distal-most tip 142. The needle holder 16 defines a needle passage 144 that is in communication with the track 120 and a distal opening 146. The distal opening 146 is offset from the longitudinal axis 56 in a forward direction.

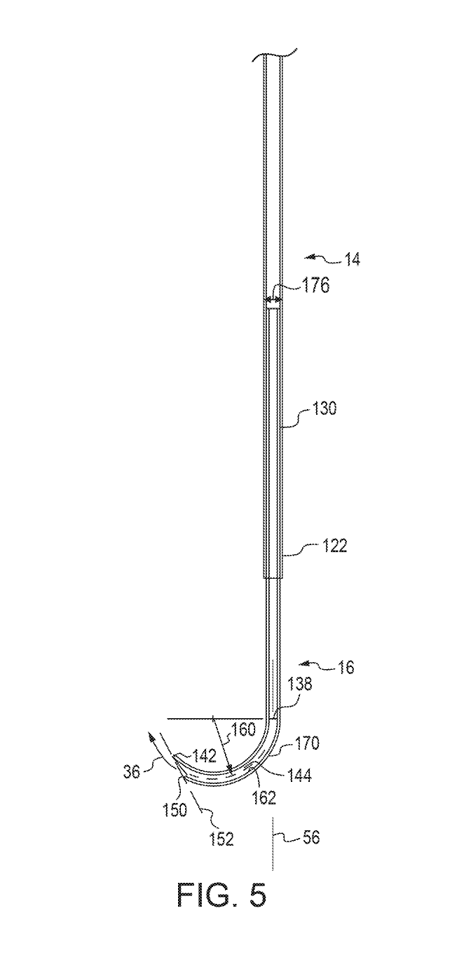

In the illustrated embodiment, the distal end section 140 of the needle holder 16 is configured to allow the second end 32 of the needle 20 to release from the needle holder 16 at a location offset from the distal-most tip 142 in a direction opposite to the advance direction 36. With reference to FIG. 5, the distal end section 140 of the needle holder 16 includes an offset edge 150 forming a part of a boundary of the distal opening 146 adjacent the location where the second end 32 of the needle 20 is released from the needle holder 16. With reference back to FIG. 3, as the needle 20 advances in the advance direction 36, the first end 30 of the needle 20 can pass through the target tissue 24. The second end 32 of the needle 20, however, need not travel past the distal-most tip 142 of the needle holder 16 before being released from the needle holder 16. Such a configuration of the distal opening 146 facilitates loading of the needle 20 and the suture 22 into the needle passage 144, which occurs by inserting the second end 32 of the needle 20 into the distal opening 146 and moving the needle 20 with respect to the needle holder 16 in a direction opposite to the advance direction 36. The configuration of the distal opening 146 also mitigates the likelihood that the first end 30 of the needle 20 may pass through the suture 22 when being passed through the target tissue 24.

The distal opening 146 is non-circular. With reference to FIG. 5, a line 152 intersects the offset edge 150 and the distal-most tip 142. This line 152 is offset from 90° with respect to a line drawn tangent to a point on the advance direction 36 where the advance direction intersects the line 152. As such, the distal opening 146 can be considered to be beveled. Even with the non-circular distal opening 146, when the needle 20 is in the retracted position (shown in FIG. 4), the first end 30 of the needle 20 is recessed inwardly (downwardly in FIG. 4) from the distal-most tip 142 within the needle passage 144. The distal-most tip 142 can also be rounded (see FIG. 1), which allows for the surgeon to grab or “hook” the target tissue 24 on the internal side 26 thereof.

With reference to FIG. 5, the needle passage 144 in the illustrated embodiment is curved and follows a curved needle passage radius 160, which is measured along a centerline of the needle passage 144. The curved needle radius 34 for the curved needle 20 depicted in FIG. 3, and the curved needle passage radius 160 are similar, but need not be identical. By not matching the curved needle radius 34 to the curved needle passage radius 160, friction between the needle 20 and the inner surface 162 of the needle holder 16 helps retain the needle 20 in the needle holder 16, for example during transport. When the needle 20 is in the retracted position, the needle 20 can be in contact with the inner surface 162 of the needle holder 16 in at least three different locations along the length, or arc length, of the needle 20.

At least a portion of the suture 22 extends along the needle passage 144 from the second end 32 of the needle 20 toward the distal opening 146 between the needle 20 and the inner surface 162 of the needle holder 16 when the needle 20 is received in the needle passage 144 and the actuator 12 is in the first operating position. By having the suture 22 extend from the distal opening 146 of the needle holder 16, a double-armed suture can be used with the suturing device 10. For example, the suture 22 can be a double-armed suture having the needle 20, which will also be referred to as the first needle, at a first end of the suture 22 and a second needle (not shown, but identical to the first needle) at a second, opposite, end of the suture 22. The first needle is loaded into the suturing device 10 and the second needle is loaded into an identical suturing device (not shown).

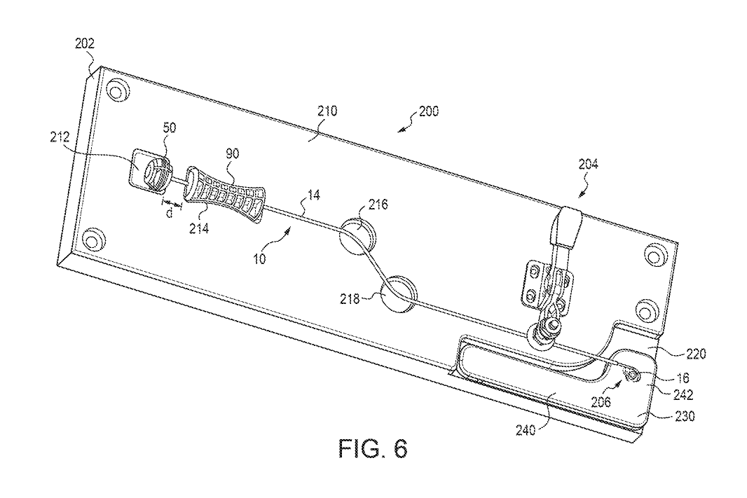

FIG. 6 depicts the suturing device 10 on a needle loading fixture 200. The needle loading fixture 200 generally includes a chassis 202, a clamp assembly 204 connected with the chassis 202, and a needle holder 206 moveably mounted to the chassis 202. The loading fixture 200 is useful for loading the needle 20 (FIG. 3) into the suturing device 10.

The chassis 202 includes a chassis surface 210. The chassis surface 210 in the illustrated embodiment is planar; however, this is not required. The chassis includes an actuator button recess 212, a handle recess 214, an upper bend recess 216, a lower bend recess 218, and a slide recess 220. With the actuator 12 (FIG. 1) appropriately positioned on the chassis 202, the actuator button recess 212 receives the button 50 and the handle recess 214 receives the handle 90. With the suturing device 10 appropriately placed on the chassis 202, an upper bend in the elongate body 14 is positioned in the upper bend recess 216 and a lower bend of the elongate body 14 is received in the lower bend recess 218. With the suturing device 10 appropriately placed on the chassis 202, the needle holder 16 extends over the slide recess 220.

The actuator button recess 212 is spaced from the handle recess 214 a distance greater than or equal to a distance the button 50 travels between an unactuated position (i.e., the first position described above) to an actuated position (i.e., the second position described above) on the suturing device 10. In other words, the actuator button recess 212 is spaced the distance d (see also FIG. 2) from the handle recess 214.

The upper bend recess 216 and the lower bend recess 218 facilitate loading the suturing device 10 on the fixture 200. The upper bend recess 216 and the lower bend recess 218 are both circular in configuration in the illustrated embodiment, and each have a diameter larger than an outer diameter 176 (FIG. 5) of the elongate body 14. More particularly, the upper bend recess 216 and the lower bend recess 218 each have a diameter at least two times that of the portion, i.e., the elongate body 14, of the suturing device 10 received in the respective recesses 216, 218.

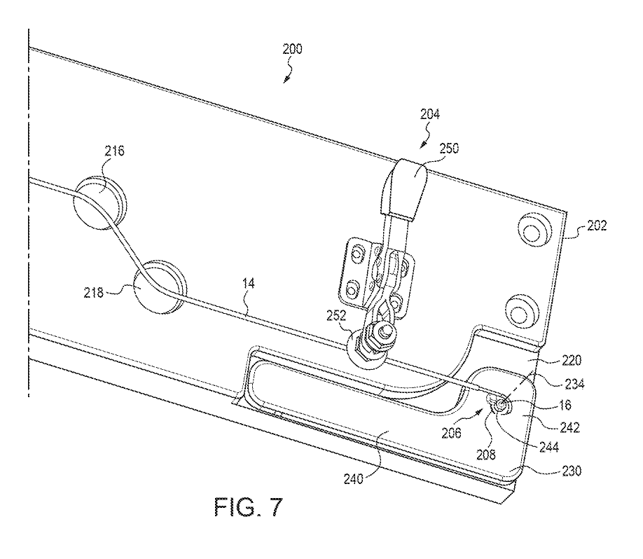

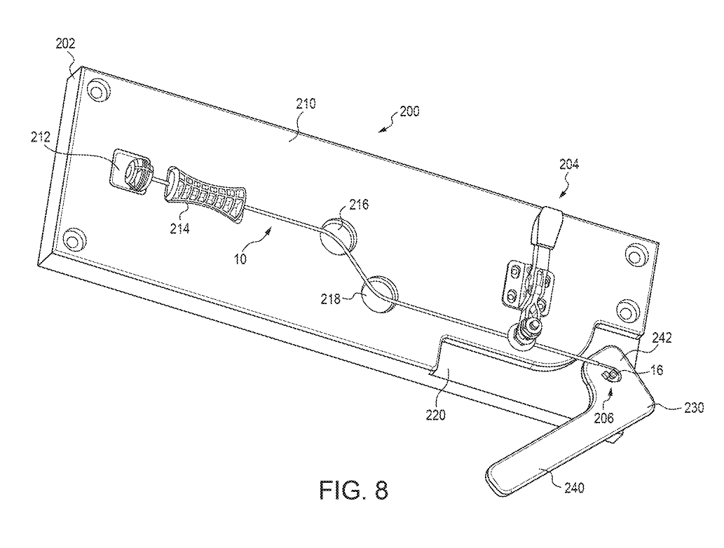

The needle loading fixture 200 also includes a slide 230. In the illustrated embodiment, the slide 230 is rotatably mounted to the chassis 202 within the slide recess 220. The needle holder 206 is mounted to the slide 230 so that the needle holder 206 rotates along with the slide 230 as the slide rotates with respect to the needle holder 16, which is fixed in place during loading of the needle 20 through the action of the clamp assembly 204. As such, the needle holder 206 is also rotatable with respect to the chassis 202. The needle holder 206 is rotatable with respect to the chassis 202 about a needle holder rotational axis 234 (FIG. 7). The needle holder 206 is offset from the needle holder rotational axis 234 a radius r, which is equal to the curved needle radius 34, which can be less than 5 mm. The needle holder 206 can include a small tube 208 that grips the needle 20 between the first end 30 and the second end 32 (FIG. 3) so as not to touch or damage the first end 30, i.e., the pointed end, of the needle 20.

The slide 230 is generally L-shaped in the illustrated embodiment. The slide 230 includes an arm section 240 and a foot section 242. With reference to FIG. 7, the slide 230 also includes a through hole (not visible) that receives an axle 244 that connects the slide 230 with the chassis 202. The axle 244 allows the slide 230 to pivot about the needle holder rotational axis 234 with respect to the chassis 202.

With continued reference to FIG. 7, the clamp assembly 204 on the loading fixture 200 can be similar to a known hold-down toggle clamp that includes a handle 250 and a pressure pad 252. The clamp assembly 204 operates in a known manner such that the pressure pad 252 contacts the elongate body 14 of the suturing device 10 to fix the location of the elongate body 14 and the remainder of the suturing device 10 with respect to the chassis 202.

A method of assembling a suturing device will be described with reference to the suturing device 10 and the loading fixture 200; however, the method of assembling the suturing device can be used to load other suturing devices and can be used with other loading fixtures (or without a loading fixture) where appropriate. The method of assembling the suturing device 10 includes holding the suturing device 10, and with reference to the illustrated embodiment, the suturing device 10 can be held with the needle loading fixture 200. The method further includes inserting the needle 20 (FIG. 3) having the suture 22 (FIG. 3) attached thereto through the distal opening 146 (FIG. 1) into the needle passage 144 (FIG. 3) of the suturing device 10. The method can further include maintaining a portion of the suture 22 extending out of the distal opening 146 and outside of the suturing device 10, such as in a manner shown in FIG. 4.

As described above and with reference to FIG. 3, the needle 20 includes a first end 30, which is pointed, and a second end 32 from which the suture 22 extends. Inserting the needle 20 can further include inserting the second end 32 of the needle 20 and folding the suture 22 such that a portion of the suture 22 extends along the needle passage 144 between the needle 20 and the inner surface 162 (FIG. 5) of the suturing device 10 defining the needle passage 144. The suture 22 is shown as folded in such a manner in FIG. 4.

The method of assembling the suturing device 10 can also include gripping the needle 20 between the first end 30 and the second end 32 with a needle holder 206 and inserting the needle 20 by moving the needle holder 206 with respect to the suturing device 10, and more particularly with respect to the needle holder 16. The needle holder 206 is moved with respect to the suturing device 10 along a plane that is coplanar with the centerline (see FIG. 5) of the needle passage 144.

When using the needle loading fixture 200, holding the suturing device 10 can include placing the suturing device 10 on the chassis 202, and retaining the suturing device 10 on the chassis 202 using the clamp assembly 204. As discussed above, the suturing device 10 includes the actuator 12 (FIG. 1) that is moveable between the first operating position and the second operating position. Movement of the actuator 12 from the first operating position toward the second operating position moves the needle 20 in the advance direction 36 with respect to the needle holder 16 thus moving the needle 20 toward the released condition, which is shown in FIG. 3. Placing the suturing device 10 on the chassis 202 can include placing the suturing device 10 in the first operating position on the chassis 202, and inserting the needle 20 can further include inserting the needle 20 with the suturing device 10 in the first operating position.

In the illustrated embodiment, the needle 20 is curved and the needle passage 144 is also curved. As such, inserting the needle 20 can further include pushing the needle 20 into the needle passage 144 until the needle frictionally engages the inner surface 162 of the suturing device 10 defining the needle passage 144. Inserting the needle can also include inserting the first end 30 of the needle 20 into the needle passage 144 such that the first end 30 is positioned inwardly from the distal-most tip 142 of the suturing device 10, such as that shown in FIG. 4.

As mentioned above, the distal end section of the suturing device 10 includes the distal-most tip 142 and the offset edge 150 (FIG. 4) forming a part of a boundary of the distal opening 146. The line 152 (FIG. 5) intersecting the offset edge 150 and the distal-most tip 142 is offset from 90° with respect to an insertion direction in which the needle 20 is inserted, and maintaining a portion of the suture 22 extending out of the distal opening 146 further includes passing the suture 22 over the offset edge 150.

As mentioned above, a double-arm suture can also be utilized. A first end of the suture can be connected with one needle, such as the needle 20 shown in FIG. 3, and a second end of the suture can be connected with another needle (not shown). The first needle can be inserted into a first needle passage of a first suturing device while a portion of the suture 22 extends out of the distal opening 146 and the second needle can be inserted into an identical suturing device (not shown) in a similar manner.

The suturing device 10 can be loaded in a factory or in or adjacent to an operating room, for example. FIG. 2 schematically depicts a sealed package 260 in which the suturing device 10 having the needle 20 loaded therein can be placed and sealed. As such, the method of assembling the suturing device 10 can including placing the suturing device 10 with the needle 20 inserted therein and the suture 22 extending out of the distal opening 146 in the package 260, and sealing the package 260 for shipment. Alternatively, the suturing device 10 can be shipped without the needle 20 loaded therein, or the suturing device 10 can be re-loaded within an operating room. As such, the method of assembling the suturing device 10 can include removing the suturing device 10 from a sealed package, such as the sealed package 260, prior to inserting the needle 20 into the needle passage 144.

A suturing device, a method of assembling the suturing device, and a loading fixture to facilitate loading a needle in the suturing device have been described above in particularity. Modifications and alterations will occur to those upon reading and understanding the preceding detailed description. The invention, however, is not limited to only the embodiments described above. Instead, the invention is broadly defined by the appended claims and the equivalents thereof. It will be appreciated that various of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A loading fixture for loading a needle into a suturing device, the loading fixture comprising:
a chassis;
a clamp assembly connected with the chassis; and
a needle holder movably mounted to the chassis.

2. The loading fixture of claim 1, wherein the chassis includes an actuator button recess and a handle recess, wherein the actuator recess is spaced from the handle recess a distance greater than or equal to a distance an actuator button travels between an unactuated position to an actuated position on the suturing device.

3. The loading fixture of claim 1, wherein the chassis includes an upper bend recess and a lower bend recess each having a diameter at least two times that of the portion of the suturing device received in the respective recess.

4. The loading fixture of claim 1, wherein the needle holder is rotatable with respect to the chassis.

5. The loading fixture of claim 4, wherein the needle holder is rotatable with respect to the chassis about a needle holder rotational axis and the needle holder is offset from the needle holder rotational axis less than 5 mm.

6. The loading fixture of claim 1, further comprising a slide rotatably mounted to the chassis, wherein the needle holder is mounted to the slide so that the needle holder rotates along with the slide as the slide rotates with respect to the needle holder.

7. The loading fixture of claim 6, wherein the chassis includes a slide recess and the slide is received in the slide recess.

8. A method of assembling a suturing device, comprising:
holding a suturing device;
inserting a needle having a suture attached thereto through a distal opening into a needle passage of the suturing device; and
maintaining a portion of the suture extending out of the distal opening and outside of the suturing device.

9. The method of claim 8, wherein the needle includes a first end, which is pointed, and a second end from which the suture extends, wherein inserting the needle further includes inserting the second end of the needle and folding the suture such that a portion of the suture extends along the needle passage between the needle and an inner surface of the suturing device defining the needle passage.

10. The method of claim 8, wherein the needle includes a first end, which is pointed, and a second end from which the suture extends, the method further comprising gripping the needle between the first end and the second end with a needle holder and inserting the needle further includes moving the needle holder with respect to the suturing device.

11. The method of claim 10, wherein inserting the needle further includes moving the needle holder with respect to the suturing device along a plane that is coplanar with a centerline of the needle passage.

12. The method of claim 8, wherein holding the suturing device further includes:
placing the suturing device on a chassis; and
retaining the suturing device on the chassis using a clamp assembly.

13. The method of claim 12, wherein the suturing device includes a movable actuator movable between a first operating position and a second operating position, wherein movement of the actuator from the first operating position toward the second operating position moves the needle in an advance direction with respect to a needle holder of the suturing device thus moving the needle toward a released condition, wherein placing the suturing device on the chassis includes placing the suturing device in the first operating position on the chassis, and inserting the needle further includes inserting the needle with the suturing device in the first operating position.

14. The method of claim 8, wherein the needle is curved and the needle passage is curved, wherein inserting the needle further includes pushing the needle into the needle passage until the needle frictionally engages an inner surface of the suturing device defining the needle passage.

15. The method of claim 8, wherein the needle includes a first end, which is pointed, and a second end opposite to the first end, wherein inserting the needle further includes inserting the first end of the needle into the needle passage such that the first end is positioned inwardly from a distal-most tip of the suturing device.

16. The method of claim 8, wherein a distal end section of the suturing device includes a distal-most tip and an offset edge forming a part of a boundary of the distal opening, wherein a line intersecting the offset edge and the distal-most tip is offset from 90 degrees with respect to an insertion direction in which the needle is inserted, wherein maintaining the portion of the suture extending out of the distal opening further includes passing the suture over the offset edge.

17. The method of claim 8, wherein the needle is a first needle including a first end, which is pointed, and a second end connected with a first end the suture, wherein inserting the needle further includes inserting the first needle into a first needle passage of the suturing device, and maintaining the portion of the suture extending out of the distal opening further includes maintaining a second needle, which is attached to a second end of the suture, outside of the first needle passage.

18. The method of claim 8, further comprising:
placing the suturing device with the needle inserted therein and the suture extending out of the distal opening in a package; and
sealing the package.

19. The method of claim 8, further comprising:
removing the suturing device from a sealed package prior to inserting the needle into the needle passage.
